(19) United States
(12) Patent Application Publication
(10) Pub. No.: US 2025/0176952 A1
(43) Pub. Date: Jun. 5, 2025

(54) FORCE MEDIATED MECHANICAL TESTING DEVICE FOR THE CHARACTERIZATION OF SOFT TISSUES

(71) Applicant: Cervalia, Inc., St. Louis, MO (US)

(72) Inventors: Rodolfo Keesey, St. Louis, MO (US); Erica Marquez, St. Louis, MO (US); Kristina Dalton, St. Louis, MO (US)

(21) Appl. No.: 18/967,808

(22) Filed: Dec. 4, 2024

Related U.S. Application Data
(60) Provisional application No. 63/605,866, filed on Dec. 4, 2023.

(51) Int. Cl. A61B 10/02; G01N 3/42

(52) U.S. Cl. CPC A61B 10/0291; G01N 3/42

(57) ABSTRACT

A testing device for determining a stiffness of a patient's tissue is provided herein that includes a probe housing, an internal probe housed in the probe housing, a load cell connected to the internal probe, and a linear-actuator connected to the load cell. The patient's tissue may be joint tissue, cervical tissue, uterine tissue, muscular tissue, and/or pulmonary tissue. The internal probe may be configured to translate within the probe housing, and the linear-actuator may be configured to move the load cell, causing translation of the internal probe within the probe housing. When the load cell is moved by the linear-actuator, the internal probe may translate within the probe housing, and a tip of the internal probe may cause indentation into a patient's tissue.

### CROSS-REFERENCE TO RELATED APPLICATIONS

The present application claims priority to U.S. Provisional Patent Application No. 63/605,866, filed Dec. 4, 2023, entitled, “FORCE MEDIATED MECHANICAL TESTING DEVICE FOR THE CHARACTERIZATION OF SOFT TISSUES,” the entire contents of which are hereby incorporated by reference.

### FIELD OF INVENTION

The present invention relates generally to a force mediated mechanical testing device. More specifically, the invention relates to a force mediated mechanical testing device that is used to characterize soft tissues (e.g., joints, cervical tissue, uterine tissue, muscular tissue, and pulmonary tissue) by measuring the mechanical stiffness of the soft tissues.

### BACKGROUND OF INVENTION

Cervical insufficiency has an incidence rate of 1-8% of the general obstetric population. It is characterized by the softening of the cervical tissue, leading to dilation of the cervix before the onset of labor. Once cervical effacement (i.e., the thinning and softening of the cervix) begins, the internal pressures of the fetus and accompanying membranes begin to emerge into the vaginal canal. At this point, emergency interventions are required to prevent negative outcomes (e.g., miscarriage or severe preterm labor, the leading cause of infant death worldwide). These interventions are time-dependent and have been shown to be more effective the earlier they are administered.

There are currently no quantitative methods to diagnose cervical insufficiency before cervical effacement occurs. Current methodology requires the visual confirmation of the cervical opening widening before interventions can be implemented. This is not only reactionary but also requires constant monitoring of a patient's pregnancy by medical professionals. Additionally, current methodology for reporting the stiffness of cervical tissue includes manual palpation, which is subjective and has been shown to be inaccurate. As such, many incidences of cervical insufficiency are missed, leading to negative outcomes occurring without credited causes.

Existing mechanical testing devices are primarily aimed at materials testing and are not used in clinical settings. Ex-vivo spherical indentation devices exist on a macro scale and are not directed to in-vivo use. Existing devices are large, difficult to maneuver, impossible to fit in or around the human body, and not rated for live tissue. Rather, these existing devices are used for extracted samples of tissue held in suspension over a loading platform.

Cervical effacement first presents as a mechanical softening of tissue and occurs prior to the dilation of the cervix, which can ultimately lead to providers missing metrics for diagnosis. By monitoring the mechanical properties of the tissue over time, providers can analyze decreasing tissue stiffness, which may be used as an indicator for early interventions. This analysis of the tissue stiffness may be achieved acutely and throughout a patient's pregnancy. Therefore, there is a need in the field for a device that quantitatively measures the stiffness of the cervical tissue in-situ. The ability to detect the change in cervical stiffness prior to cervical shortening will aid in earlier treatment intervention, and therefore, a reduction in negative outcomes. Such a device may be particularly relevant and useful for reproductive health providers and clinicians as well as researchers who study biomechanics and associated properties (in one embodiment, specifically related to cervical mechanics and properties).

### SUMMARY OF INVENTION

The present invention overcomes many of the shortcomings and limitations of the prior art devices discussed above. The invention described herein includes several embodiments of a force mediated mechanical testing device.

The device as set forth herein may be used to measure the internal, mechanical properties of soft tissues via mechanical indentation. The device may measure data associated with the tissue and may store the data for later processing and review. The device consists of a load cell, a linear-actuator, and internal probe coupled in series to transfer the resistance force of the tissue displaced by the probe to the load cell directly.

The device may include a microcontroller which may control operation of the linear-actuator, and thus, movement of the internal probe. When in use, the internal probe may extend out of the device for indentation into a patient's tissue. The microcontroller may store data associated with the position of the internal probe and the force of the load cell. Such data points may be used to assess and calculate the stiffness of the patient's tissue.

### BRIEF DESCRIPTION OF DRAWINGS

FIG. 1 is a front perspective view of a force mediated mechanical testing device constructed according to the teachings of the present application;

FIG. 2 is a cross-sectional view of the device of FIG. 1; and

FIG. 3 is a rear perspective view of the device of FIG. 1.

While the disclosure is susceptible to various modifications and alternative forms, a specific embodiment thereof is shown by way of example in the drawings and will herein be described in detail. It should be understood, however, that the drawings and detailed description presented herein are not intended to limit the disclosure to the particular embodiment disclosed, but to the contrary, the intention is to cover all modifications, equivalents, and alternatives falling within the spirit and scope of the present disclosure as defined by the appended claims.

### DETAILED DESCRIPTION

The invention will now be described with reference to the drawing figures, in which like reference numerals refer to like parts throughout. For purposes of clarity in illustrating the characteristics of the present invention, proportional relationships of the elements have not necessarily been maintained in the drawing figures.

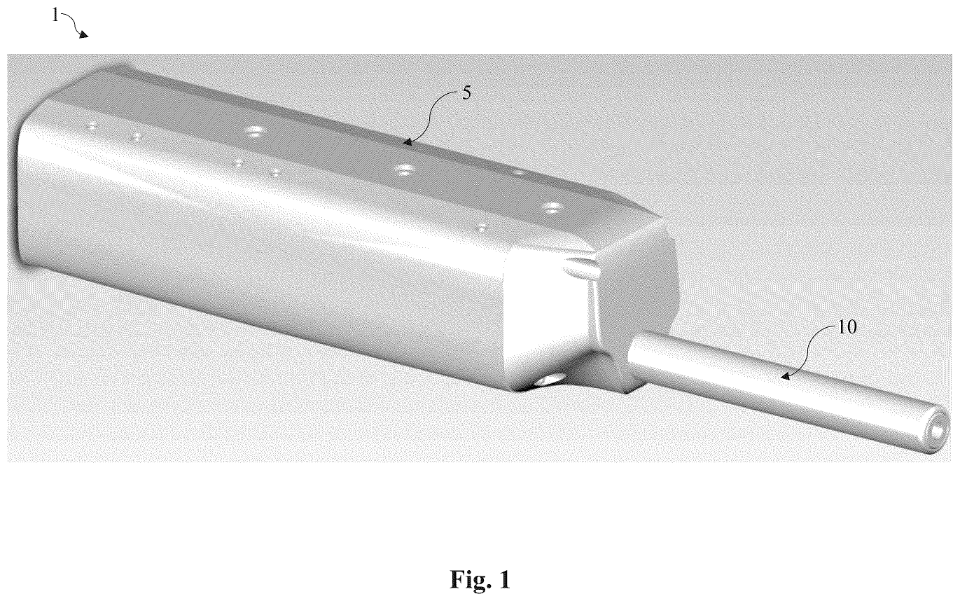

Turning first to FIG. 1, a force mediated mechanical testing device 1 may be used to measure and test the stiffness of a particular tissue. The tissue may be any relevant tissue of a patient, including, but not limited to, a patient's joint tissue, cervical tissue, uterine tissue, muscular tissue, and pulmonary tissue. In one embodiment, the device 1 is used to measure stiffness of a patient's cervical tissue. In such an instance, the device 1 may be inserted into the patient's vaginal canal to access the cervical tissue. In one embodiment, the device 1 is used to test a particular joint tissue for a patient. In such an instance, the device 1 may be pressed (or inserted via an incision) into the patient's tissue. The device 1 may include an external housing 5 to house components of the device 1. In one embodiment, the external housing 5 may be designed to fit in an operator's hand. The device 1 may include a probe housing 10, which may be designed for insertion in-situ.

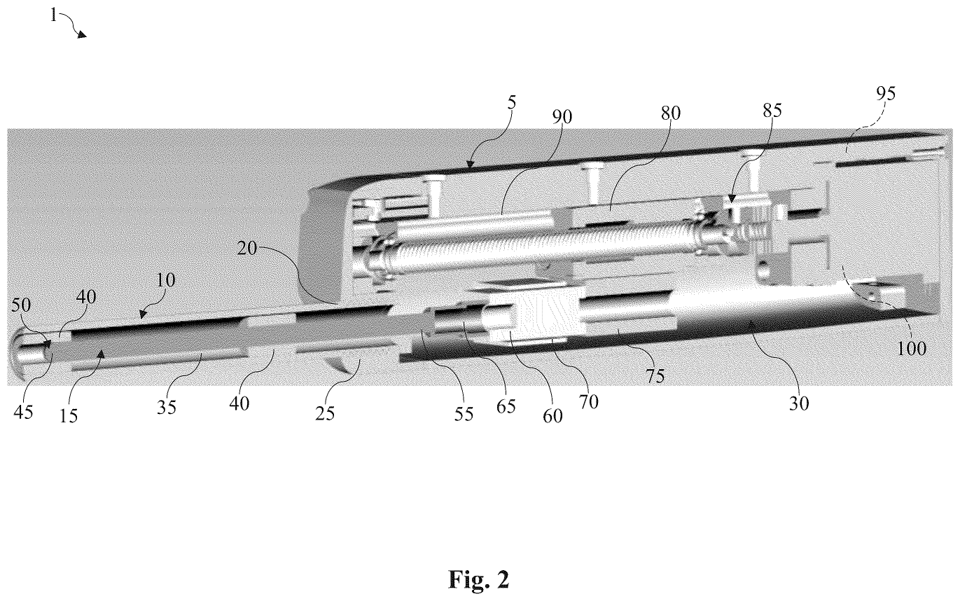

Turning to FIG. 2, the probe housing 10, which may be adjustable for various uses, may house an internal probe 15. The internal probe 15 may be any suitable length. The length of the internal probe 15 may be selected based on the intended usage of the device 1. For example, the internal probe 15 may be a particular length suitable for insertion to test the stiffness of a patient's cervical tissue. In one embodiment, the internal probe 15 is designed for controlled displacement of a patient's tissue (e.g., cervical tissue). An inner end 20 of the probe housing 10 may be threaded such that it may attach to the external housing 5. In one embodiment, the threading of the inner end 20 may mate with a threaded portion 25 in an interior 30 of the external housing 5. The inner end 20 may thread to the threaded portion 25 of the external housing 5 to create a seal. Such attachment may allow for easy replacement of both the probe housing 10 and the internal probe 15 in order to accommodate different patients using the same device 1. Although a threading mechanism is described herein, it should be understood that any form of connection or attachment may be used.

The probe housing 10 may include a channel 35 extending therethrough such that the internal probe 15 may move (e.g., slide) within the probe housing 10. The probe housing 10 may include several linear bearings 40, and the internal probe 15 may be positioned within the linear bearings 40, which may allow the internal probe 15 to adjust and move (e.g., slide) within the channel 35 through the linear bearings 40. The linear bearings 40 may aid in minimizing friction caused by movement of the internal probe 15. Such configuration may be useful to reduce noise in measurements calculated by the internal probe 15, which ultimately may aid in increasing accuracy of data collected.

As discussed above, the internal probe 15 may be designed for controlled displacement of tissue. This may be achieved via a rounded (or spherical) tip 45 on a first end 50 of the internal probe 15. Although discussed herein as a rounded or spherical shape, it should be understood that the tip 45 may be any suitable shape. The rounded tip 45 may aid in controlling the tissue displacement when the internal probe 15 is being used to test a patient's tissue. The rounded tip 45, internal probe 15, and probe housing 10 may be any suitable diameter. In one embodiment, the diameter may be selected based on the intended usage of the device 1. For example, when used to test the stiffness of a patient's cervical tissue, the diameter may be selected such that the probe housing 10 may fit within the vaginal canal to access the cervical tissue and such that the internal probe 15 and rounded tip 45 can access the cervical tissue for testing. For other intended uses, the associated diameters may be selected as suitable for such uses. The internal probe 15 may be made of stainless steel. In one embodiment, the probe housing 10 serves as external sheath to the internal probe 15 and may be made of stainless steel.

According to various embodiments, a second end 55 of the internal probe 15 may be attached to a load cell 60 via an attachment portion 65. The load cell 60 may be housed in a carriage 70, which may be mounted to a linear rail 75. The carriage 70 and its attachment to the linear rail 75 may reduce friction and constrain motion of the load cell 60. The load cell 60 may be fixed to a movement portion 80 of a linear-actuator 85, which may account for adjustment within the probe housing 10. A body 90 of the linear-actuator 85 may be fixed to the external housing 5 via a custom bracket or connection. The inline assembly of the probe housing 10, load cell 60, and linear-actuator 85 may aid in allowing the internal probe 15 to move linearly within the probe housing 10.

The linear-actuator 85 may cause the internal probe 15 to move and adjust within the channel 35 of the probe housing 10 by moving and adjusting the load cell 60 connected to the linear-actuator 85. As such, when the load cell 60 moves based on the linear-actuator 85, the internal probe 15 also may move. In one embodiment, the internal probe 15 may translate longitudinally within the channel 35 (e.g., from and toward the patient's tissue). The translation of the internal probe 15 may be sufficient such that, when retracted, the rounded tip 45 may be received in the probe housing 10 and, when extended, the rounded tip 45 may extend out of the probe housing 10. When extended, the rounded tip 45 may be extended into a patient's tissue.

According to various embodiments, the device 1 may include a microcontroller 95 which may be used to direct the linear-actuator 85 to move a specified distance. The microcontroller 95 may be used to both control the operation of the linear-actuator 85 and to log the position of the internal probe 15 and the force of the load cell 60. The linear-actuator 85 may output a position signal, which may be used for closed loop control by a motor driver 100 of the linear-actuator 85 and for the microcontroller 95 to record displacement of the internal probe 15. The measured displacement of the internal probe 15 and the resultant resistance force against the patient's cervical tissue may allow for calculation of the stiffness of the patient's tissue.

In one embodiment, the linear-actuator 85 may provide a voltage signal which may allow for substantially constant monitoring of the position of the internal probe 15. According to various embodiments, the linear-actuator 85 may include a motor driver 100. In one embodiment, the motor driver 100 may be used to convert a pulse width modulated signal (which may denote a desired displacement) to motor commands required to direct the linear-actuator 85. The linear-actuator 85 may include a position feedback sensor. The feedback sensor may be wired in parallel to the motor driver 100 and an onboard analog-to-digital converter within the microcontroller 95. According to various embodiments, the motor driver 100 may be driven by a custom printed circuit board (PCB). The linear-actuator 85 may include a linear induction sensor, which may provide feedback from the linear-actuator 85 to the microcontroller 95. It should be understood that any suitable or desirable configuration (including those now known and those hereafter developed) may be used to monitor the position of the internal probe 15. The configurations as described herein and as contemplated may allow for a real-time position of the internal probe 15 may be recorded and displayed.

When the device 1 is used and the internal probe 15 extends into the patient's tissue, the load cell 60 may measure and record the force in the microcontroller 95. Typically, soft cervical tissues may include a low force resistance, and the internal environment requires limited force to be applied in order to prevent tissue damage. To aid in ensuring that the optimal measurement of the resistive force is measured, the signal from the load cell 60 may be sent to a load cell amplifier which may include an onboard 16-bit analog-to-digital converter. The load cell amplifier may convert the measured analog signal to a digital signal, and the digital signal may then be passed to the microcontroller 95 for recording.

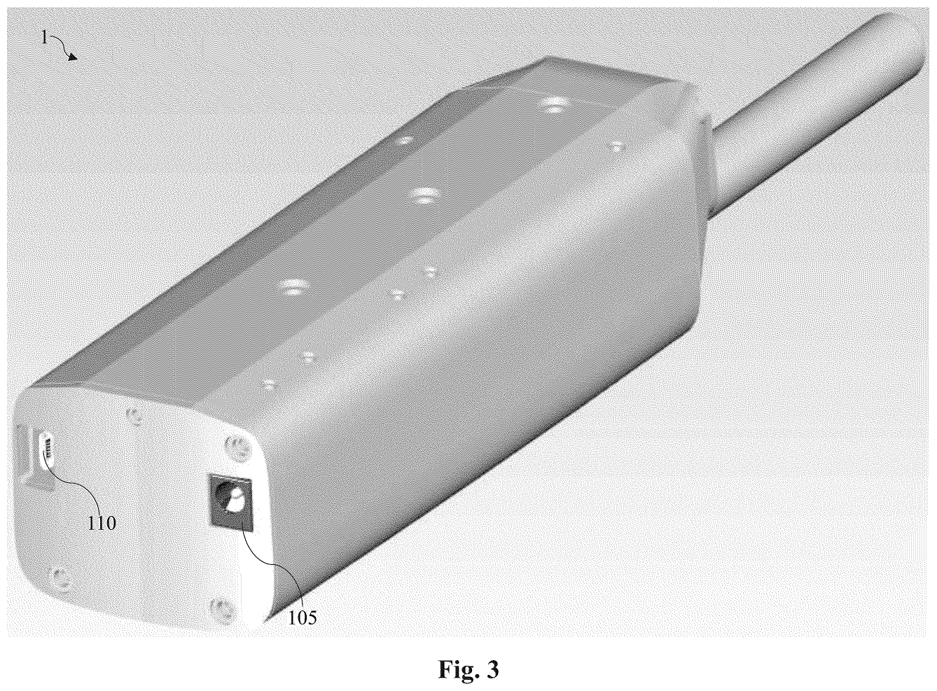

Turning now to FIG. 3, the device 1 and associated components may be powered in any suitable manner (e.g., via battery). In one embodiment, the device 1 is powered by a 12V power barrel connected to the device via a power barrel connector 105, and the power barrel may provide power to the load cell 60 and linear-actuator 85. The device 1 may include a USB port 110 from the microcontroller 95 which may allow the device 1 to be connected to a computer. Although described as a physical connection, it should be understood that any suitable connection mechanism may be included (e.g., Bluetooth). When connected, the data and measurements gathered and calculated may be transmitted to the computer. In one embodiment, the data may be transmitted in real-time to the computer, and the computer may display to an operator the position of the internal probe 15 and the resultant force as a measure of time.

According to various embodiments, the device 1 may output data in any suitable manner (including as a digitized voltage), and such data may be exported to other software for quantification and post processing. The software may convert the data into Newtons/gram force using previously-established methods. Using this conversion, the Hertzian Contact equation for spherical indentation may be used to calculate the elastic modulus (a measure of mechanical stiffness of the material, e.g., cervical tissue).

As is evident from the foregoing description, certain aspects of the present invention are not limited by the particular details of the examples illustrated herein, and it is therefore contemplated that other modifications, applications, variations, or equivalents thereof, will occur to those skilled in the art. Many such changes, modifications, variations, and other uses and applications of the present constructions will, however, become apparent to those skilled in the art after considering the specification and the accompanying drawings. In addition, unless mention was made above to the contrary, it should be noted that all of the accompanying drawings are not to scale. All such changes, modifications, variations, and other uses and applications which do not depart from the spirit and scope of the present inventions are deemed to be covered by the inventions which are limited only by the claims which follow.

1. A testing device for determining a stiffness of a patient's tissue comprising:
a probe housing;
an internal probe housed in the probe housing and configured to translate within the probe housing;
a load cell connected to the internal probe; and
a linear-actuator connected to the load cell and configured to move the load cell, causing translation of the internal probe within the probe housing;
wherein when the load cell is moved by the linear-actuator, the internal probe is configured to translate within the probe housing; and
wherein the patient's tissue is at least one of a patient's joint tissue, cervical tissue, uterine tissue, muscular tissue, and pulmonary tissue.

2. The testing device of claim 1, wherein a tip of the internal probe is substantially rounded.

3. The testing device of claim 1, wherein when the testing device is used, the probe housing abuts tissue, and the internal probe is configured to indent tissue.

4. The testing device of claim 1, further comprising a microcontroller.

5. The testing device of claim 4, wherein the microcontroller is configured to control operation of the linear-actuator.

6. The testing device of claim 4, wherein the microcontroller measures a position of the internal probe and a force of the load cell.

7. The testing device of claim 1, wherein the internal probe is connected to a plurality of linear bearings.

8. The testing device of claim 7, wherein the plurality of linear bearings reduce friction caused by translation of the internal probe within the probe housing.

9. A testing device for determining a stiffness of a patient's tissue comprising:
an internal probe housed in a probe housing and configured to translate within the probe housing; and
a linear-actuator configured to cause translation of the internal probe;
wherein when the linear-actuator is activated, the internal probe is configured to translate within the probe housing; and
wherein the patient's tissue is at least one of a patient's joint tissue, cervical tissue, uterine tissue, muscular tissue, and pulmonary tissue.

10. The testing device of claim 9, further comprising a load cell connected at a first side to the internal probe and at a second side to the linear-actuator.

11. The testing device of claim 9, wherein the internal probe comprises a tip, and wherein the tip is substantially rounded.

12. The testing device of claim 9, wherein when the testing device is used, the internal probe is configured to indent patient tissue.

13. The testing device of claim 9, further comprising a microcontroller.

14. The testing device of claim 13, wherein the microcontroller is configured to control operation of the linear-actuator.

15. The testing device of claim 13, wherein the microcontroller measures a position of the internal probe and a force of a load cell.

16. The testing device of claim 9, wherein the internal probe is connected to a plurality of linear bearings, and wherein the plurality of linear bearings reduce friction caused by translation of the internal probe within the probe housing.

17. A testing device for determining a stiffness of a patient's tissue comprising:
a probe housing comprising an internal probe and a plurality of linear bearings, wherein the internal probe is configured to translate within the probe housing through the plurality of linear bearings;
a load cell connected to the internal probe;
a linear-actuator connected to the load cell and configured to move the load cell, causing translation of the internal probe within the probe housing; and
a microcontroller configured to control operation of the linear-actuator;
wherein when the load cell is moved by the linear-actuator, the internal probe is configured to translate within the probe housing; and
wherein the patient's tissue is at least one of a patient's joint tissue, cervical tissue, uterine tissue, muscular tissue, and pulmonary tissue.

18. The testing device of claim 17, wherein the internal probe comprises a tip, and wherein the tip is substantially rounded.

19. The testing device of claim 17, wherein the internal probe is configured to indent patient tissue.

20. The testing device of claim 17, wherein the microcontroller measures a position of the internal probe and a force of the load cell.
